(19) United States
(12) Patent Application Publication
(10) Pub. No.: US 2012/0321621 A1
(43) Pub. Date: Dec. 20, 2012

(54) INHIBITION OF FcyR-MEDIATED PHAGOCYTOSIS WITH REDUCED IMMUNOGLOBULIN PREPARATIONS

(76) Inventor: Donald R. Branch, Toronto (CA)

(21) Appl. No.: 13/595,662

(22) Filed: Aug. 27, 2012

Related U.S. Application Data
(62) Division of application No. 12/544,619, filed on Aug. 20, 2009.
(60) Provisional application No. 61/136,233, filed on Aug. 20, 2008.

(30) Foreign Application Priority Data
Aug. 20, 2008 (CA) 2639003

(51) Int. Cl. A61K 39/395; A61P 25/00; A61P 37/00; A61P 19/02; C12N 5/02; A61P 7/06

(52) U.S. Cl. 424/133.1; 424/141.1; 435/375; 424/130.1

(57) ABSTRACT

The invention relates to pharmaceutical compositions for inhibiting FcγR-mediated phagocytosis that include a reduced immunoglobulin inhibitor of FcγR-mediated phagocytosis, such as anti-D, in combination with a pharmaceutically acceptable carrier. The invention also relates to methods for treating or preventing an autoimmune or alloimmune disease that include administering to a subject in need thereof a therapeutically effective amount of a reduced immunoglobulin inhibitor of FcγR-mediated phagocytosis.

### CROSS REFERENCE TO RELATED APPLICATIONS

This application is a divisional of U.S. patent application Ser. No. 12/544,619, filed on Aug. 20, 2009, which itself claims priority to U.S. Provisional Patent Application Ser. No. 61,136,233, filed on Aug. 20, 2008, and Canadian Patent Application Serial No. 2,639,003, filed on Aug. 20, 2008. The disclosure of each of these applications is incorporated by reference in its entirety.

### TECHNICAL FIELD

The present invention relates to reduced immunoglobulin inhibitors of FcγR-mediated phagocytosis, and to pharmaceutical compositions containing the same, intended for treatment or prevention of autoimmune or alloimmune diseases. The invention also relates to methods for treating autoimmune and alloimmune diseases.

### BACKGROUND

Intravenously administered immunoglobulin preparations, intravenous immune globulin (WIG) and anti-D, are commonly used to treat various conditions including immune thrombocytopenic purpura (ITP; El-Shanawany T, Sewell W A, Misbah S A, Jolles S. Current clinical uses of intravenous immunoglobulin. Clin Med 2006; 6:356-9; Darabi K, Abdel-Wahab O, Dzik W H. Current usage of intravenous immune globulin and the rationale behind it: the Massachusetts General Hospital data and a review of the literature. Transfusion 2006; 46:741-53; Bussel J. Treatment of immune thrombocytopenic purpura in adults. Semin Hematol 2006; 43 (3 Suppl 5):S3-10; Kjaersgaard M, Hasle H. A review of anti-D treatment of childhood idiopathic thrombocytopenic purpura. Pediatr Blood Cancer 2006; 47:717-20; Tarantino M D, Young G, Bertolone S J, et al. Single dose of anti-D immune globulin at 75 micrograms/kg is as effective as intravenous immune globulin at rapidly raising the platelet count in newly diagnosed immune thrombocytopenic purpura in children. J Pediatr 2006; 148:489-94; Aledort L M, Salama A, Kovaleva L, et al. Efficacy and safety of intravenous anti-D immunoglobulin (RHOPHYLAC®; CSL Behring AG Corporation, Bern, Switzerland) in chronic immune thrombocytopenic purpura. Hematology 2007; 12:289-95).

ITP is an autoimmune disease characterized by Fcγ receptor (FcγR)-mediated clearance of autoantibody sensitized platelets (Darabi K et al. op. cit.; Bussel J. op. cit.; Crow A B, Lazarus A. Role of Fcgamma receptors in the pathogenesis and treatment of idiopathic thrombocytopenic purpura. J Pediatr Hematol Oncol 2003; 25:S14-8; Ware R E, Zimmerman S A. Anti-D: mechanisms of action. Semin Hematol 1998; 35 (1 Suppl 1):14-22; Bussel J B. Fc receptor blockade and immune thrombocytopenic purpura. Semin Hematol 2000; 37:261-6; Kjaersgaard M, Aslam R, Kim M, et al. Epitope specificity and isotype of monoclonal anti-D antibodies dictate their ability to inhibit phagocytosis of opsonized platelets. Blood 2007; 110:1359-61).

Although immunoglobulin therapy can be effective, there are many disadvantages to the use of immunoglobulins. For example, IVIG is extremely expensive where 1 to 2 g per kg of body weight is administered several times per treatment regimen and 1 g can cost up to $100 (Stasi R, Provan D. Management of immune thrombocytopenic purpura in adults. Mayo Clin Proc 2004; 79:504-22; Cowden J, Parker S K. Intravenous immunoglobulin: production, uses and side effects. Pediatr Infect Dis J 2006; 25: 641-2). Anti-D is produced from pooled human sera, and this can result in world wide shortages of anti-D (Cowden J, ibid.; Milgrom H. Shortage of intravenous immunoglobulin. Ann Allergy, Asthma & Immunol 1998:81:97-100), not only because of the demand for its use in treatment of ITP, but also because it is used to prevent hemolytic disease of the newborn due to sensitization of an Rh-negative mother by an Rh⁺ fetus (Rhesus disease). In addition, each batch of anti-D and IVIG has the potential of harboring pathogens (Power J P, Davidson F, O'Riordan J, Simmonds P, Yap P L, Lawlor E. Hepatitis C infection from anti-D immunoglobulin. Lancet 1995; 346:372-3; Reichl H E, Foster P R, Welch A G, et al. Studies on the removal of a bovine spongiform encephalopathy-derived agent by processes used in the manufacture of human immunoglobulin. Vox Sang 2002:83:137-45), particularly those where no detection method yet exists, such as the infectious agent causing vCJD disease (Reichl H E, et al. ibid.) Moreover, anti-D and IVIG have been shown to have severe and even fatal adverse effects (Gaines A R. Acute onset hemoglobinemia and/or hemoglobinuria and sequelae following Rh(o)(D) immune globulin intravenous administration in immune thrombocytopenic purpura patients. Blood. 2000; 95(8):2523-9; Christopher K, Horkan C, Barb I T, Arbelaez C, Hodgdon T A, Yodice P C. Rapid irreversible encephalopathy associated with anti-D immune globulin treatment for idiopathic thrombocytopenic purpura. Am J Hematol 2004; 77:299-302; Gaines A R. Disseminated intravascular coagulation associated with acute hemoglobinemia or hemoglobinuria following Rh(0)(D) immune globulin intravenous administration for immune thrombocytopenic purpura. Blood. 2005; 106(5):1532-7; Sekul E, Cupler E J, Dalakas M C. Aseptic meningitis associated with high-dose intravenous immunoglobulin therapy: frequency and risk factors. Ann Intern Med 1994; 121:259-62; Go R S, Call T G. Deep venous thrombosis of the arm after intravenous immunoglobulin infusion: case report and literature review of intravenous immunoglobulin-related thrombotic complications. Mayo Clin Proc 2000; 75:83-5; Dalakas M C. High-dose intravenous immunoglobulin and serum viscosity: risk of precipitating thromboembolic events. Neurology 1994; 44:223-6; Elkayam O, Paran D, Milo R, Davidovitz Y, Almoznino-Sarafian D, Zeltser D, Yaron M, Caspi D. Acute myocardial infarction associated with high dose intravenous immunoglobulin infusion for autoimmune disorders: a study of four cases. Ann Rheum Dis 2000; 59:77-80; Woodruff R K, Grigg A P, Firkin F C, Smith I L. Fatal thrombotic events during treatment of autoimmune thrombocytopenia with intravenous immunoglobulin in elderly patients. Lancet 1986; 2:217-8). The inflated costs of immunoglobulins can be attributed to these factors.

Therefore, it would be beneficial to improve current treatment or develop new treatments for ITP and other immune cytopenias that would be more cost-efficient and have decreased associated risks. If the amount of anti-D used in the treatment of ITP could be significantly reduced, this could further diminish the side effects and alleviate some of the concerns over the use of this immunoglobulin.

### SUMMARY

In one aspect, the invention provides a pharmaceutical composition for inhibiting FcγR-mediated phagocytosis comprising a reduced immunoglobulin inhibitor of FcγR-mediated phagocytosis in combination with a pharmaceutically acceptable carrier.

In another aspect, the invention provides a method for treating or preventing an autoimmune or alloimmune disease comprising administering to a subject in need thereof a therapeutically effective amount of a reduced immunoglobulin inhibitor of FcγR-mediated phagocytosis.

In yet another aspect, the invention provides a method for inhibiting tissue destruction due to an autoimmune disease comprising administering to a subject in need thereof a therapeutically effective amount of a reduced immunoglobulin inhibitor of FcγR-mediated phagocytosis.

In still another aspect, the invention provides an immunoglobulin preparation comprising reduced anti-D or reduced IVIG for inhibiting FcγR-mediated phagocytosis.

In another aspect, the invention provides a method for inhibiting FcγR-mediated phagocytosis in a FcγR-expressing phagocytic cell comprising exposing said phagocytic cell to a reduced immunoglobulin inhibitor of FcγR-mediated phagocytosis.

In another aspect, the invention provides a method for increasing inhibitory activity of an immunoglobulin inhibitor of FcγR-mediated phagocytosis comprising subjecting said immunoglobulin inhibitor of FcγR-mediated phagocytosis to disulfide reduction.

### BRIEF DESCRIPTION OF THE DRAWINGS

FIG. 1 illustrates the ability of anti-D and IVIG to block FcγR-mediated phagocytosis. Immunoglobulins were used at different concentrations to first block FcγRs on Mφ, the Mφ were washed, anti-D-coated RBCs were overlaid, and phagocytosis was determined and compared to an untreated control. (A) Titration of slide-rapid tube anti-D (n=8) starting at a dilution of 1 in 3. (B) Titration of WINRHO® SDF (Cangene Corporation, Winnipeg, Manitoba, Canada) anti-D (n=3) starting at 1×10⁻³ mg per mL (0.001 mg/mL). (C) Titration of IVIG (n=9) starting at 1.0 mg per mL. Graphs A and C are representative of at least three independent experiments. Graph B is a single experiment. Standard error of the mean is represented by error bars.

FIG. 2. Chemically treated anti-D has enhanced ability to inhibit FcγR-mediated phagocytosis. Thimerosal and anti-D were used alone or in combination to determine the effect on ability to inhibit FcγR-mediated phagocytosis after extensive dialysis. (A) Mean percentage inhibition of phagocytosis by undialyzed thimerosal used at 10⁻⁵ mol per L (n=9), dialyzed thimerosal at 10⁻⁵ mol per L (n=9) and 10⁻³ mol per L (n=9), slide-rapid tube anti-D at 1 in 6 dilution (n=9), and anti-D at 1-in-6 dilution that had been mixed with thimerosal at 10⁻⁵ mol per L (n=9) and 10⁻³ mol per L (n=9) for 24 hours before dialysis. Each bar represents three combined independent experiments and error bars represent SEM. *p=0.0005; **p=0.0004. (B) Mean percentage inhibition of phagocytosis by undialyzed thimerosal used at 10⁻⁵ mol per L (n=3), dialyzed thimerosal at 10⁻⁵ mol per L (n=3), WINRHO® SDF anti-D at 0.000025 μg per mL (n=3), or 0.00001 μg per mL (n=3) and WINRHO® SDF anti-D at these concentrations that had been treated with 10⁻⁵ mol per L thimerosal (n=3). *p=0.003; **p=0.0386. These results are representative of two independent experiments. (C) An identical experiment as in A but with anti-Kell-sensitized rr,KK RBCs. Anti-D-sensitized R₂R₂ RBCs were also used as a control for the phagocytic readout (n=3 for each point). *p=0.0002.

FIG. 3. FACS analysis of the effect of treatment of anti-D with thimerosal on monocyte viability and apoptosis.

(A) With the THP-1 monocyte cell line, effects of untreated or thimerosal-treated anti-D were assessed with dual-color flow cytometry with PI and annexin V-FITC. Tests utilized untreated THP-1 or THP-1 treated with dialyzed slide-rapid tube anti-D alone, used at a 1-in-6 dilution, or dialyzed anti-D that had been previously mixed with thimerosal at 10⁻⁵ or 10⁻³ mol per L. Annexin V-FITC fluorescence is represented on the horizontal axis and PI fluorescence is shown on the vertical axis. The viable, early apoptotic, late apoptotic, and necrotic cells are found in the lower left, lower right, upper right, and upper left quadrants, respectively. Percentage of cells within each quadrant is indicated. (B and C) With primary PBMNC-derived adherent monocytes, effects of untreated or thimerosal-treated anti-D were assessed with three-color flow cytometry with anti-CD14-APC, PI, and annexin V-FITC. Tests utilized untreated primary monocytes or primary monocytes treated with dialyzed WINRHO® SDF anti-D at a concentration of 0.0005 μg per mL, used alone, or previously mixed with thimerosal at 10⁻⁵ mol per L.

(B) Results with total ungated adherent cells;

(C) results when only CD14+ monocytes are evaluated. Annexin V-FITC fluorescence is represented on the horizontal axis and PI fluorescence is shown on the vertical axis. The viable, early apoptotic, late apoptotic, and necrotic cells are found in the lower left, lower right, upper right, and upper left quadrants, respectively. Percentage of cells within each quadrant is indicated.

FIG. 4. Treatment of IVIG with thimerosal has no significant effect on its ability to inhibit antibody-mediated in vitro phagocytosis and results in no cell toxicity. (A) Mean percentage inhibition of in vitro phagocytosis by dialyzed (from left to right): thimerosal at 10⁻⁵ mol per L (n=9) or 10⁻³ mol per L (n=9), IVIG at 0.01 mg per mL (n=9), IVIG (0.01 mg/mL) that had been mixed with thimerosal at 10⁻⁵ mol per L (n=3) or 10⁻³ mol per L (n=6), IVIG at 0.05 mg per mL (n=11), and IVIG (0.05 mg/mL) that had been mixed with thimerosal at 10⁻⁵ mol per L (n=6) or 10⁻³ mol per L (n=9). Error bars represent the means±SEM. (B) Tests showing FACS results for untreated THP-1, THP-1 treated with dialyzed IVIG at 0.05 mg per mL, or dialyzed IVIG that had been previously mixed with thimerosal at 10⁻³ mol per L.

FIG. 5. Chemical structures of thimerosal, dithiothrietol (DTT), and p-toluenesulfonylmethyl mercaptan.

FIG. 6. Ability of DTT-modified anti-D to block FcγR-mediated phagocytosis. Anti-D before (1) and after dialysis (2), DTT before (3) and after dialysis (4) and anti-D modified with DTT after dialysis (5) are compared to an untreated control by percent inhibition. Results represent the mean+/− standard error of the mean (SEM) of 6 independent experiments.

FIG. 7A. Ability of p-toluenesulfonylmethyl mercaptan-modified anti-D to block FcγR-mediated phagocytosis. Anti-D before (1) and after dialysis (2), p-toluenesulfonylmethyl mercaptan after dialysis (3) and anti-D modified with p-toluenesulfonylmethyl mercaptan after dialysis (4) are compared to an untreated control by percent inhibition. Results represent the mean+/− standard error of the mean (SEM) of 6 independent experiments.

FIG. 7B. Effect of reduction and S-alkylation on ability of anti-D to inhibit phagocytosis. p-toluenesulfonylmethyl mercaptan was used at 10⁻²M, 10⁻⁵M and 10⁻⁶M to reduce disulfide bonds within anti-D. 5 mM iodoacetamide was used to S-alkylate the reduced disulfide bonds to prevent re-oxidation. Reduced only and reduced +S-alkylated anti-D were used to block FcγR and compared to an untreated control by percent inhibition of phagocytosis. Results represent the mean+/− SEM of 3 independent experiments. *p=0.000028; **p=0.000007.

FIG. 8. FACS analysis of the effect of treatment of anti-D with DTT and p-toluenesulfonylmethyl mercaptan. The toxicity of chemically-modified anti-D was tested using primary PBMNC-derived adherent monocytes after incubation with untreated, DTT- or p-toluenesulfonylmethyl mercaptan-treated anti-D. Effects of treatment were assessed with dual-color flow cytometry with PI and annexin V-FITC. Tests utilized untreated monocytes and monocytes treated with dialyzed slide-rapid tube anti-D alone, used at a 1-in-6 dilution, or dialyzed anti-D that had been previously mixed with DTT or p-toluenesulfonylmethyl mercaptan at 10-2 mol per L. Annexin V-FITC fluorescence is represented on the horizontal axis and PI fluorescence is shown on the vertical axis. The viable, early apoptotic, late apoptotic, and necrotic cells are found in the lower left, lower right, upper right, and upper left quadrants, respectively. Percentage of cells within each quadrant is indicated.

### DETAILED DESCRIPTION

It has been recently shown that certain chemical compounds containing para-nitrophenyl and sulfur-reactive substituent groups can inhibit FcγR-mediated phagocytosis in vitro and may pose promising drug candidates for future treatment of immune cytopenias (Rampersad G, Suck G, Sakac D, Fahim S, Foo A, Denomme G A, Langler R F, Branch D R. Chemical compounds that target thiol/disulfide groups on mononuclear phagocytes inhibit immune mediated phagocytosis of red blood cells. Transfusion 2005; 45:384-93; Foo A H, Fletcher S P, Langler R F, Porter C H, Branch D R. Structure-function studies for in vitro chemical inhibition of Fc gamma receptor-mediated phagocytosis. Transfusion 2007; 47:290-8). The mechanism of action of these compounds has been proposed to involve indirect interference of the interaction of the FcγR with antibody-coated cells by steric hindrance after binding to thiol groups on the surface of monocyte-macrophages (Mφ) within close proximity to FcγRs (Rampersad G, et al. op. cit.). Immunoglobulins, in contrast, have been shown to inhibit FcγR interaction with antibody-coated cells by directly binding to the FcγR resulting in “blockade” of this interaction (Crow A B, op. cit.; Bussel J B. 2000 op. cit.; Lazarus A H, Crow A R. Mechanism of action of IVIg and anti-D in ITP. Transfus Apher Sci 2003; 28:249-55).

Since those compounds reported to inhibit FcγR-mediated phagocytosis by a steric mechanism have the potential to react with all proteins throughout an in vivo system, the possibility of targeting these chemical compounds to Mφ FcγR with immunoglobulins as carrier molecules has been previously suggested (Rampersad, G. C., op. cit.). If combining a phagocytosis-inhibiting chemical compound with an immunoglobulin that interacts specifically with FcγRs were to obtain an additive or synergistic effect on the ability of the chemically treated immunoglobulin to inhibit FcγR-mediated phagocytosis in vitro, this approach could enhance the efficacy of immunoglobulin therapies and would have the potential to be translated into in vivo use resulting in lower dosage with reduced cost and side effects.

Presented herein are in vitro results with a prototype compound, thimerosal, previously reported to strongly inhibit in vitro phagocytosis (Rampersad, G. C., et al. ibid.) and shown to bind irreversibly to anti-D (Shulman I A, Branch C A, Nitsun M, Gallagher M T, Branch D R. Thimerosal inhibition of monocyte-macrophage Fc-receptor (FcR) function. Blood 1986; 68 (Suppl 1):86a). For proof-of-concept, anti-D or WIG have been treated with thimerosal to determine whether the chemically treated immunoglobulins have significantly enhanced efficacy to inhibit FcγR-mediated phagocytosis in vitro.

Also presented herein are in vitro studies with reduced immunoglobulin inhibitors of FcγR-mediated phagocytosis, which are discussed in further detail below, following the results and discussion of the studies with thimerosal-treated anti-D.

### Cells, Chemicals, and Immunoglobulins for Thimerosal Studies

The human monocyte cell line THP-1 (ATCC 202, Manassas, Va.) was maintained in continuous culture in RPMI 1640 (Gibco/Invitrogen, Burlington, Ontario, Canada) containing 10 percent fetal bovine serum (FBS; Sigma-Aldrich, Oakville, Ontario, Canada) and 0.1 percent gentamycin (Gibco/Invitrogen) at 37° C. and 5 percent CO₂. THP-1 is a nonadherent leukemia cell line that is phagocytic and contains FcγRs but no cytoplasmic immunoglobulins (Tsuchiya S, Yamabe M, Yamaguchi Y, Kobayashi Y, Konno T, Tada K. Establishment and characterization of a human acute monocytic leukemia cell line (THP-1). Int J Cancer 1980; 26:171-6). Normal human peripheral blood was obtained from volunteers after informed consent as per a Canadian Blood Services Research Ethics Board-approved Protocol 2005.002. Thimerosal was purchased from BioShop Canada, Inc. (Burlington, Ontario, Canada). Anti-Kell and homozygous (KK), D− (rr) red blood cells (RBCs) were a gift from T. Frame (Immucor, Atlanta, Ga.). D+ (R₂R₂) RBCs were provided by the Canadian Blood Services. Human polyclonal anti-D for slide and tube reagent tests (slide-rapid tube) was a gift of M. Moulds (Immucor, Houston, Tex.). Anti-D (WINRHO® SDF) was obtained from Cangene (Winnipeg, Manitoba). WIG (human) therapy (Gammagard S/D, Baxter, Deerfield, Ill.) was a gift from A. Lazarus (Canadian Blood Services, Toronto, Ontario, Canada). Anti-CD14 conjugated to allophycocyanin (APC), immunoglobulin G (IgG)2b, κ, isotype control (anti-dansyl), and fluorescein isothiocyanate (FITC)—goat anti-mouse were purchased from BD Bioscience PharMingen (Mississauga, Ontario, Canada).

Chemical Treatment of Immunoglobulin with Thimerosal

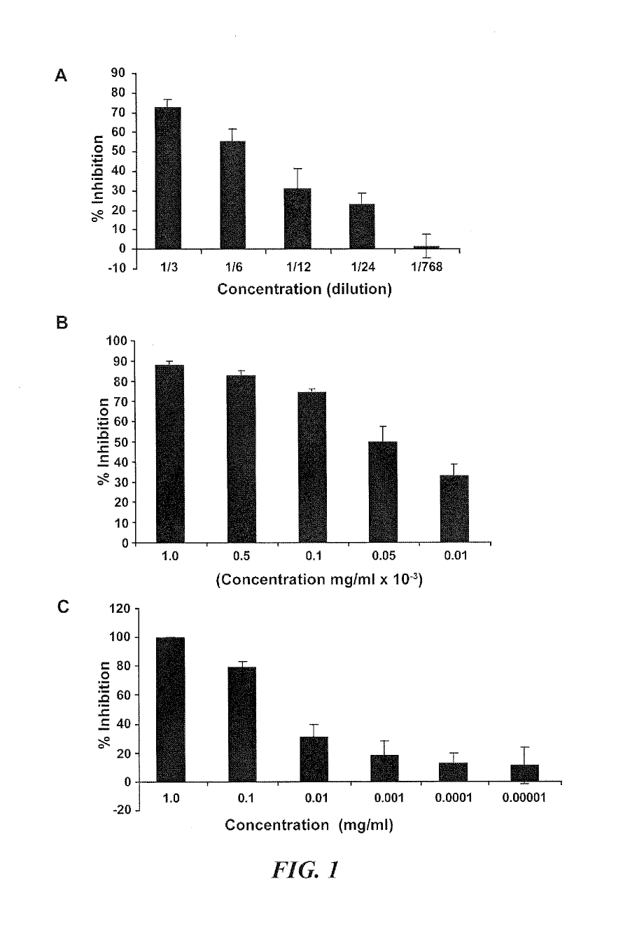

The test concentration of immunoglobulin (anti-D or IVIG) was chosen after titration of each immunoglobulin on its own for its dose response to inhibit FcγR and phagocytosis of anti-D-coated RBCs with a monocyte monolayer assay (MMA) as previously described (Rampersad, G. C., et al. op. cit.; Foo, A. H. et al. op. cit.). Based on these dose-response inhibitory titration curves, a concentration for each immunoglobulin was chosen so as to be close to a half-maximum (50%) inhibitory effect. These concentrations were a 1-in-6 dilution for slide-rapid tube anti-D, 0.025×10⁻³ mg per mL for WINRHO® SDF anti-D, and 0.05 mg per mL for WIG (FIG. 1). For each experiment, the controls, which consisted of chemical alone and immunoglobulin alone, and a mixture of chemical and immunoglobulin in phosphate buffered saline (PBS), pH 7.4, were placed in separate tubes (Sarstedt, Nümbrecht, Germany) and gently rotated for 24 hours at room temperature. The solutions were then transferred to respective cellulose dialysis tubing membranes with a molecular size cutoff of 12,000 Da (Sigma-Aldrich) that had been cut to length and washed as per the manufacturer's directions by boiling in a solution containing 1 mmol per L ethylenediaminetetraacetic acid (BioShop) and 2 percent sodium bicarbonate (Fisher Scientific, Ottawa, Ontario, Canada) in PBS. Dialysis was performed in large beakers of PBS for 5 to 7 days at 4° C. with daily changes of PBS. After dialysis, the MMA was utilized for comparison of an unmanipulated control (see below) to the ability of each preparation to inhibit FcγR-mediated phagocytosis of anti-D- or anti-Kell-coated R₂R₂ or rr,KK RBCs, respectively. Also, the MMA was used to determine whether free thimerosal had dialyzed out of the tubing, rendering it incapable to of inhibiting phagocytosis compared to undialyzed thimerosal. Furthermore, the effect of thimerosal-treated immunoglobulin was compared to immunoglobulin alone on the ability to inhibit Mφ phagocytosis.

RBC Sensitization and MMA Assay with Thimerosal-Treated Anti-D

Preparation of anti-Kell-sensitized rr,KK RBCs and anti-D-sensitized R₂R₂ RBCs has been previously described (Rampersad, G. C., et al. op. cit.; Foo, A. H. et al. op. cit.). Briefly, R₂R₂ or rr,KK RBCs were resuspended in PBS to 5 percent concentration, mixed with an equal volume of human polyclonal anti-D (Immucor) or anti-Kell, respectively, in PBS solution, and then incubated for 1 hour at 37° C. and 5 percent CO₂, after which the sensitized RBCs were washed four times in PBS and resuspended to 2.5 percent in PBS. Before the RBC suspension was mixed with an equal volume of culture medium (RPMI 1640, Gibco/Invitrogen) supplemented with 10 percent (vol/vol) FBS (Sigma-Aldrich) and 20 mmol per LHEPES buffer, pH 7.4 (Gibco/Invitrogen), an indirect antiglobulin test was performed to assess the level of antibody coating of the RBCs and yielded a 4+ reaction.

The MMA was performed as previously described with slight modification (Foo, A. H. et al. ibid.). Briefly, after attachment of monocytes to coverslips, the coverslips were washed vigorously in PBS to remove any nonadherent cells, including RBCs, and then overlaid with immunoglobulin alone diluted in PBS (with or without dialysis), thimerosal-treated immunoglobulin diluted in PBS that had been extensively dialyzed, and undialyzed thimerosal used alone. PBS alone was used as the positive control for maximum phagocytosis that all preparations were compared against. After 1 hour of incubation at 37° C. and 5 percent CO₂, the coverslips were washed again, and 1 mL of sensitized RBC suspension was layered onto the coverslip and then incubated for 2 hours at 37° C. and 5 percent CO₂ (Rampersad, G. C., et al. op. cit.; Foo, A. H. et al. op. cit.).

### Statistical Analysis

A phagocytic index was calculated as previously described (Rampersad, G. C., et al. op. cit.; Foo, A. H. et al. op. cit.; Branch D R, Gallagher M T, Mison A P, Sy Siok Hian A L, Petz L D. In vitro determination of red cell alloantibody significance using an assay of monocyte-macrophage interaction with sensitized erythrocytes. Br J Haematol 1984; 56:19-29) as the unitless number of antibody-sensitized RBCs phagocytosed per 100Mφ. Typical phagocytic indices for anti-D-sensitized R₂R₂ RBCs were 80 to 90 whereas anti-Kell-sensitized rr,KK RBCs typically gave a phagocytic index of 8 to 10. Residual and phagocytosed RBCs were distinguished by relative differences in refracted light under phase-contrast microscopy. Percentage inhibition was calculated as previously described (Rampersad, G. C., et al. op. cit.; Foo, A. H. et al. op. cit.; Branch D R et al. op. cit.) taking the phagocytic control index, with only PBS, to be the maximum phagocytosis possible. The means and standard error of the mean (SEM) of the results from several independent experiments were determined and analyzed statistically. Significance of inhibition between treated and untreated Mφ were analyzed with at test and analysis of variance (ANOVA), and/or general linear model analysis and Student-Newman Keuls test. A p value of less than 0.05 was considered to be significant.

Fluorescence-Activated Cell Sorting (FACS) Analysis for Viable and Apoptotic Cells Following Incubation with Anti-D and Thimerosal-Treated Anti-D

Fluorescence-activated cell sorting (FACS) analysis (flow cytometry) and a TACS annexin V-FITC apoptosis detection kit (R&D Systems, Minneapolis, Minn.) were used to determine whether treatment with anti-D, IVIG, or chemically treated immunoglobulins exert effects on viability or apoptosis of monocytic THP-1 cells or primary peripheral blood mononuclear cell (PBMNC)-derived adherent monocytes. For THP-1, cells were collected by centrifugation (250×g for 5-10 min) and resuspended to 1×10⁵ to 1×10⁶ cells per sample in medium for controls and treatment for tests. Cells were incubated with dialyzed anti-D alone or thimerosal-treated anti-D for 1 hour at 37° C. and 5 percent CO₂ before being washed three times in PBS at 250×g for 5 to 10 minutes. Cells were then resuspended in culture medium and incubated for 2 hours before being washed in cold PBS and collected by centrifugation. Each sample was resuspended gently in 100 μL of cold annexin V incubation reagent (10 μL 10× binding buffer [R&D Systems], 0.8 μL annexin V-FITC [R&D Systems], 79.2 μL of autoclaved deionized H₂O) and incubated in the dark for 15 minutes at room temperature. Minutes before analysis by flow cytometry (FACS), 10 μL of propidium iodide (PI; R&D Systems) was added to each sample. FACS was performed with two-color analysis on a flow rate-calibrated (by use of BD CALIBRITE™ beads, Becton Dickinson, San Jose, Calif.) flow cytometer (FACSCALIBUR™ E4795, Becton Dickinson) and its accompanying software for data analysis (CELLQUEST™, Becton Dickinson).

For PBMNC-derived monocytes, monocyte monolayers were prepared as for the standard MMA assay, and dialyzed anti-D or thimerosal-treated anti-D was incubated with the monocyte monolayer for 4 hours. The monocytes were then scraped from the plates with a rubber policeman, washed in cold PBS, and collected by centrifugation. Each sample was resuspended gently in 100 μL of cold anti-CD14 conjugated to APC (BD Biosciences PharMingen) and incubated for 30 minutes on ice followed by gentle washing and then incubation with cold annexin V incubation reagent and PI as described for THP-1 cells. FACS was performed on ungated cells and cells gated on the CD14+ monocyte population with three-color flow cytometry. Isotype control for CD14− APC was mouse IgG_{2bk} (anti-dansyl; BD Biosciences PharMingen).

### Effect of Thimerosal Treatment of Anti-D on Efficacy to Inhibit Phagocytosis

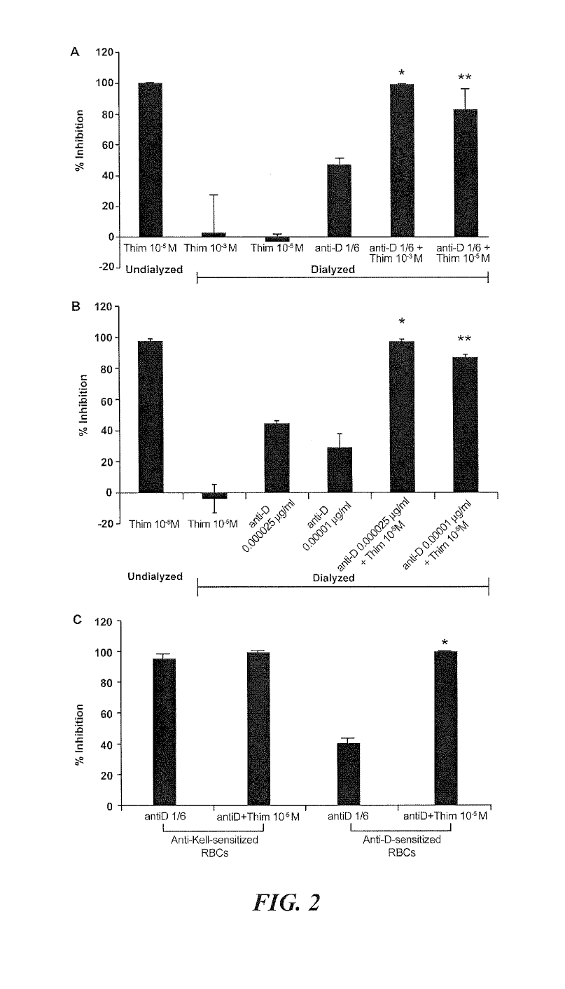

Thimerosal at 10⁻⁵ or 10⁻³ mol per L was mixed with slide-rapid tube anti-D used at a 1-in-6 dilution or WINRHO® SDF anti-D used at two different concentrations of 0.025×10⁻³ and 0.01×10⁻³ mg per mL to give an approximate 50 percent inhibitory activity on phagocytosis (FIG. 1). As shown in FIG. 2, with the chemical treatment of immunoglobulin protocol, both anti-D preparations at the concentrations tested maintained their ability to inhibit phagocytosis in vitro by approximately 40 to 50 percent after dialysis. Anti-D at the same concentration, but that had been treated with thimerosal at 10⁻⁵ or 10⁻³ mol per L, was able to inhibit phagocytosis by approximately 83 percent (p=0.0005) and 100 percent (p=0.0004), respectively, after dialysis with slide-rapid tube anti-D (FIG. 2A) and by approximately 97 percent (p=0.003) and 89 percent (p=0.0386) for WINRHO® SDF anti-D (FIG. 2B). The significant difference in efficacy between anti-D alone and chemically treated anti-D was not attributed to effects of free thimerosal as evident in FIG. 2. Although undialyzed thimerosal used alone at 10⁻⁵ or 10⁻³ mol per L (data not shown) inhibits phagocytosis by 100 percent (FIG. 2), it no longer inhibits phagocytosis after dialysis (FIG. 2) indicating any free thimerosal had been removed from the dialysis tubing and hence the sample.

### Ability of Anti-D Induced FcγR Blockade to Inhibit Phagocytosis of Anti-Kell-Sensitized D− RBCs

Because the experiments described utilize anti-D to inhibit subsequent phagocytosis of anti-D-sensitized RBCs, it was questioned whether the inhibitory activity may be enhanced due to the use in the assay of D+ RBCs and whether or not the FcγR blockade observed when using anti-D could be generalized to other blood group antigens: To address this, D− homozygous Kell RBCs were sensitized with anti-Kell and these cells were used to monitor the effect of anti-D blocking of phagocytosis. A similar ability for anti-D to block FcγR-mediated phagocytosis was observed when using anti-Kell to sensitize rr,KK RBCs (FIG. 2C); however, in this experiment, the FcγR blocking activity of anti-D used alone at a dilution of 1 in 6 was much more effective than when using R₂R₂ RBCs sensitized with anti-D (approx. 95 percent inhibition of the phagocytosis of the anti-Kell-sensitized rr cells), possibly due to the much lower phagocytic index of anti-Kell-sensitized RBCs compared to anti-D-sensitized RBCs. Thus, although an increase to 100 percent blockade of the phagocytosis of anti-Kell-sensitized RBCs was observed when using thimerosal-treated anti-D, this was not significant. Nevertheless, this experiment clearly demonstrates the ability of anti-D to inhibit phagocytosis of anti-Kell-sensitized D− RBCs providing evidence that anti-D-mediated inhibition of antibody-mediated phagocytosis in vitro is unrelated to the target cell or antibody used.

### Toxicity of Thimerosal-Treated Anti-D on THP-1 Cells

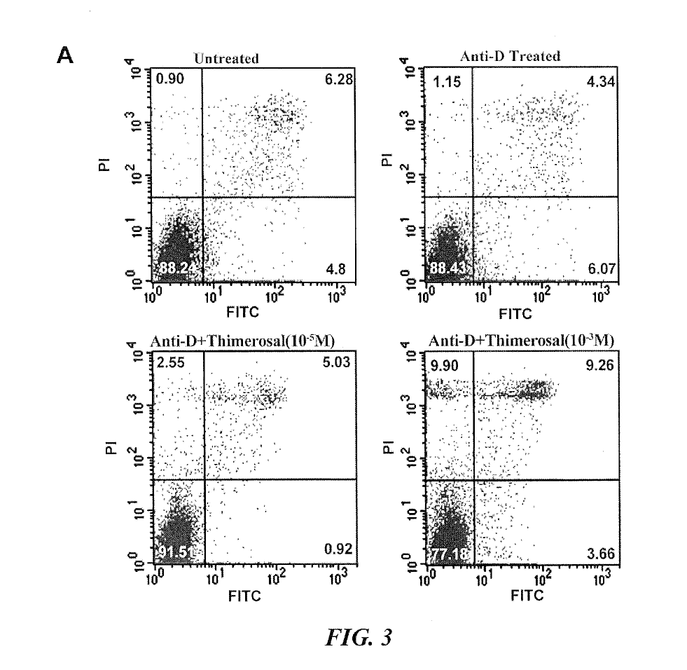

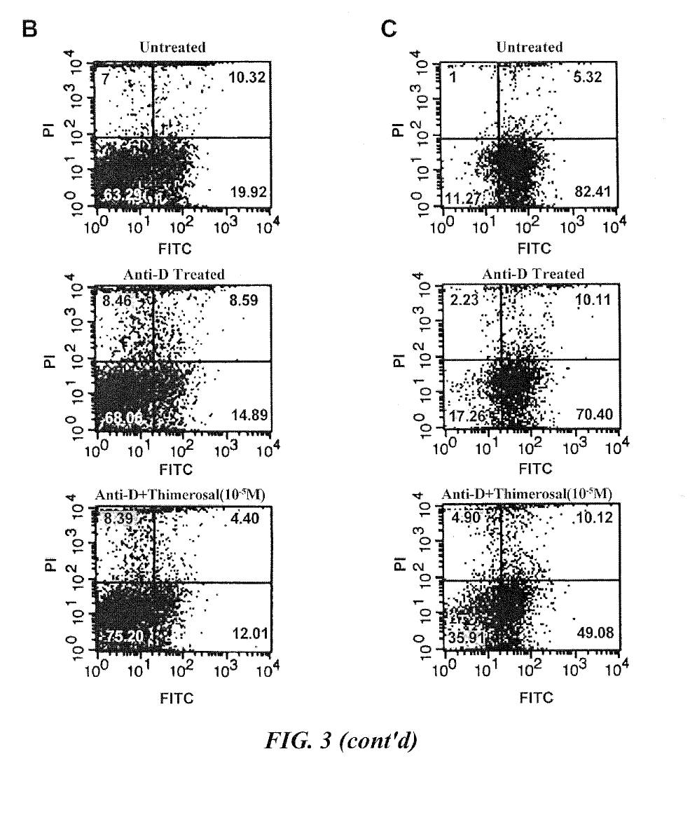

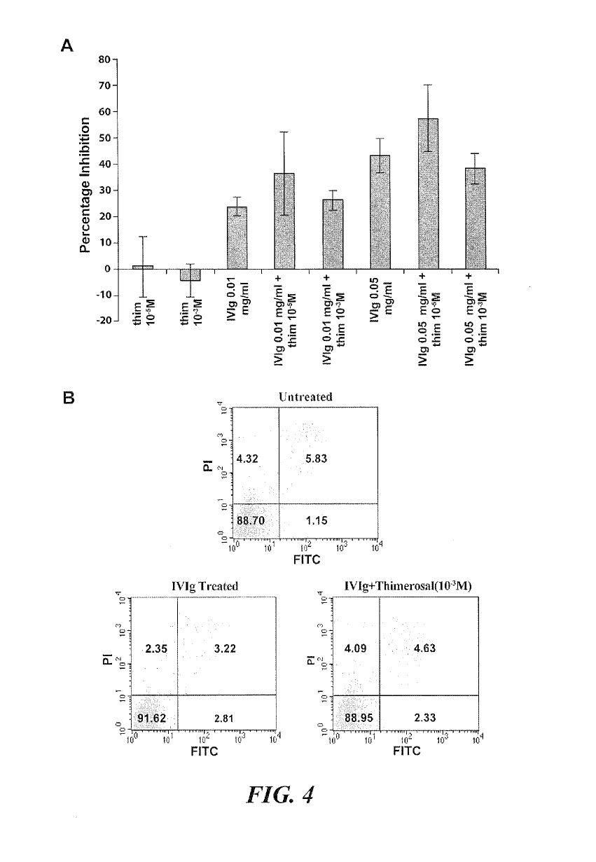

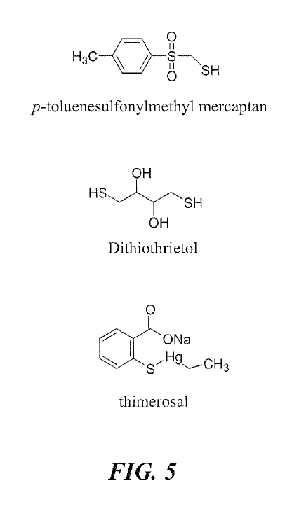

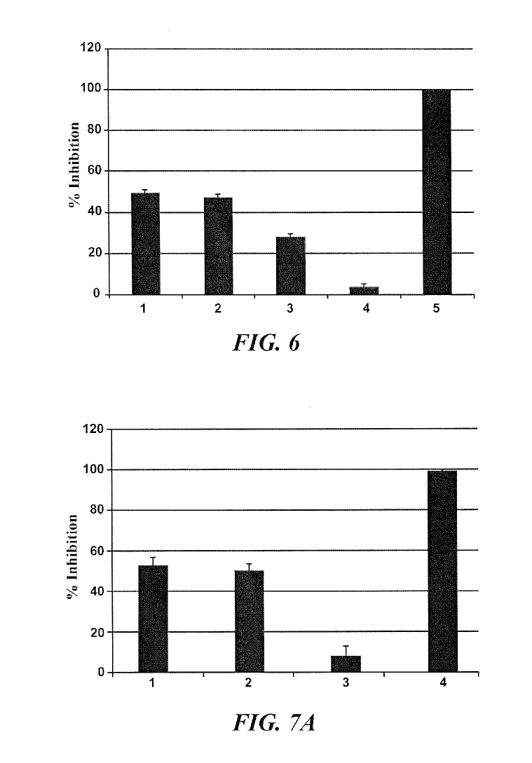

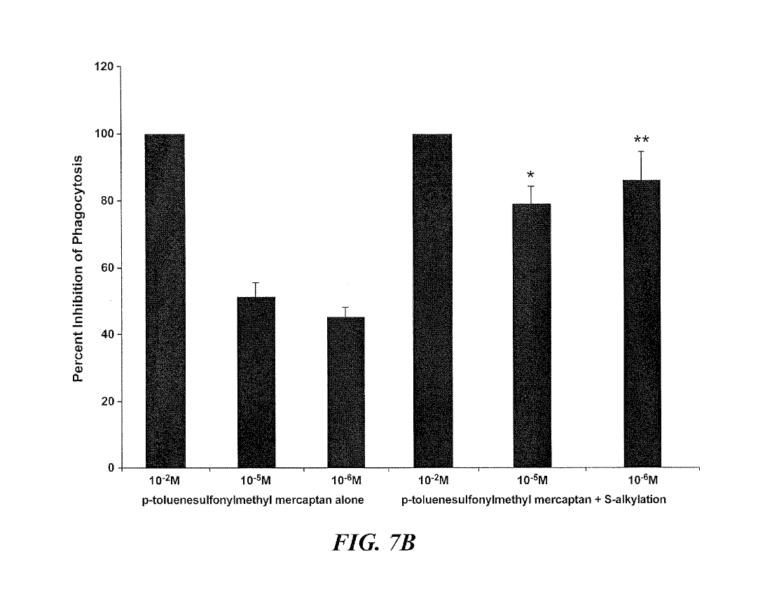

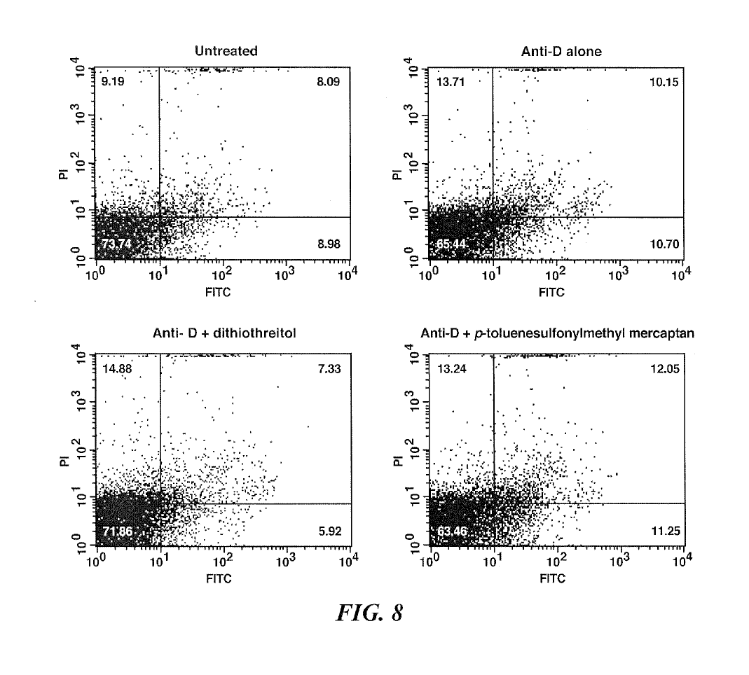

Chemically treating different preparations of anti-D with thimerosal enhanced the ability of anti-D to inhibit in vitro phagocytosis by up to 100 percent (FIG. 2). It was necessary, however, to rule out the possibility that this effect was attributed to enhanced cell death as a result of the treatment. Therefore, FACS analysis and annexin V-FITC apoptosis detection kit (R&D Systems) were used to evaluate the effect of anti-D and thimerosal-treated anti-D on human monocytic THP-1 cell viability and apoptosis. THP-1 cells were used for these studies because they are a nonadherent monocyte cell line (Tsuchiya S, et al. op. cit.) allowing for ease of and reproducible FACS analyses, and these cells also constitutively express both the high affinity receptor for IgG, FcγRI, and FcγRIIA, both involved in phagocytosis (Auwerx J, Staels B, Van Vaeck F, Ceuppens J L. Changes in IgG Fc receptor expression induced by phorbol 12-myristate 13-acetate treatment of THP-1 monocytic leukemia cells. Leuk Res 1992; 16:317-27). Given the viable cell counts of untreated and treated cells, it was determined that treatment with anti-D at a 1-in-6 dilution or anti-D that had been mixed with 10⁻⁵ mol per L thimerosal did not significantly alter cell viability or apoptosis (FIG. 3A). It was noted, however, that when anti-D was mixed with thimerosal at 10⁻³ mol per L and used to treat THP-1 cells, there was a 12.5 percent decrease in viable cell count compared to untreated THP-1 cells (FIG. 3A, lower right-hand panel, lower left-hand quadrant). This was not sufficient, however, to explain the much larger increase (twofold) in the ability of the 10⁻³ mol per L thimerosal-treated anti-D to inhibit FcγR-mediated phagocytosis.

### Toxicity of Thimerosal-Treated Anti-D on Primary Monocytes

Because THP-1 cells are a long-term-derived cell line, which may be more resistant to the toxic effects of thimerosal-treated anti-D, toxicity testing was also performed with primary PBMNC-derived adherent monocytes. After removing the treated adherent monocytes, with three-color FACS analysis, it was possible to examine annexin V binding and PI staining on the total ungated cell population (FIG. 3B) and also on the gated CD14+ cell population (FIG. 3C) with a fluorescent-labeled anti-CD14. It can be seen that thimerosal-treated anti-D has little effect on apoptosis or cell death when using either ungated or anti-CD14− gated cells (FIGS. 4B,4C) consistent with the THP-1 results. It was unexpected to see two populations of adherent monocytes, CD14+ and CD14− (FIG. 3B compared to FIG. 3C). Moreover, the CD14+ cell population appears to be undergoing apoptosis even for the untreated cells, perhaps due to the scraping off of the cells from the plates to perform the FACS analysis. Nevertheless, there was no increased apoptosis apparent when using anti-D or thimerosal-treated anti-D. Indeed, it appears that when using thimerosal-treated anti-D that this treatment may result in less apoptosis and more viable CD14+ cells (FIG. 3C, bottom panel, lower left-hand quadrant).

### Effect of Thimerosal Treatment of IVIG on Efficacy to Inhibit Phagocytosis

In contrast to the results of chemical treatment of anti-D, the efficacy of thimerosal-treated IVIG did not statistically exceed that of IVIG used alone (FIG. 4A). This experiment was repeated several times for IVIG at concentrations of 0.01 and 0.05 mg per mL and thimerosal concentrations ranging from 10⁻⁵ to 10⁻³ mol per L (FIG. 4A). Although the inhibitory activity of WIG alone and chemically treated IVIG did not significantly differ according to t test, ANOVA, general linear model, or Student-Newman-Keuls t tests, a trend of enhanced ability of IVIG to inhibit phagocytosis in vitro after chemical treatment was observed (FIG. 4A; E. Vidgen, Department of Biostatistics, University of Toronto, personal communication, 2006).

### Toxicity of Thimerosal-Treated IVIG on THP-1 Cells

Chemically treating WIG with thimerosal did not result in a significant enhancement of the ability of WIG to inhibit in vitro phagocytosis (FIG. 4A). Because a slight increase of the inhibitory activity of the chemically treated IVIG was observed compared to untreated IVIG, investigations were carried out to determine whether or not thimerosal treatment of IVIG results in any toxic effects on THP-1 cell viability and/or apoptosis that could explain this slight enhancement. With FACS analysis and annexin V-FITC apoptosis detection kit (R&D Systems), the viable cell counts of untreated and treated cells were comparable (FIG. 4B). Hence, it was determined that treatment with 0.05 mg per mL IVIG or IVIG that had been mixed with thimerosal at 10⁻³ mol per L did not significantly alter cell viability or apoptosis.

Discussion of Results Obtained with Thimerosal-Treated Anti-D

Novel or improved treatments for immune cytopenias, such as ITP, are needed to overcome the numerous disadvantages of the use of immunoglobulins, anti-D and IVIG (Cowden, J. et al. op. cit.; Milgrom, H. op. cit.; Gaines A R, 2000 op. cit.; Gaines A R, 2005 op. cit.; Sekul, E. et al. op. cit.; Go R S, et al. op. cit.; Dalakas, M C op. cit.; Elkayam, O. et al. op. cit.; Woodruff, R K et al. op. cit.) Severe and even fatal side effects have been reported (Gaines, A R, 2005 op. cit.) and both IVIG and anti-D are processed from human source material and, thus, have a slight but real risk of infectious disease transmission (Cowden, J. et al. op. cit.; Sekul et al. op. cit.; Siegel J. Safety considerations in IGIV utilization. Int Immunopharmacol 2006; 6:523-7). A small-molecular-weight drug-based approach to the treatment of immune cytopenias, including ITP, would be cost-efficient to manufacture, relatively easy to produce in large amounts, and free of risk of disease transmission. The inventor has previously investigated and identified potential drug candidates for use in inhibiting Mφ FcγR-mediated phagocytosis in vitro (Rampersad G. et al. op. cit.; Foo, A H et al. op. cit.). Rampersad and coworkers (Rampersad G. et al. op. cit.) showed that chemicals that can react with sulfur moieties on macrophage membranes can effectively block phagocytosis of antibody-sensitized RBCs. Further investigation of the mechanism of action of the lead compounds from these initial studies, with structure-function analyses, identified the crucial minimum structural requirements for an optimal inhibitory effect as a thiol-reactive substituent group and apara-nitrophenyl group (Foo, A H et al. op. cit.).

Although this initial work implies that drug-based approaches could have utility in vivo, it became apparent that drugs containing sulfur-reactive groups would have the potential to react with many different proteins in the blood and tissues, and this could result in adverse effects and/or loss of efficacy if these drugs were ever used in vivo. Therefore, it was speculated that one might be able to bind these candidate drugs to immunoglobulins currently being used for the treatment of ITP to target these compounds to the Mφ, responsible for the removal of the antibody-coated blood cells in these disorders, via their FcγR. It was further speculated that by binding the drug candidates to anti-D and/or IVIG that one might also be able to increase the efficacy of these immunoglobulins to inhibit the phagocytosis of antibody-coated blood cells due to an additive or synergistic effect of this combination of agents that, individually, inhibit FcγR-mediated phagocytosis. This novel approach, if successful, has the potential, if further developed with optimized drug candidates, to reduce the immunoglobulin therapy-related concerns of cost, supply, and adverse events.

Thimerosal was chosen for proof-of-concept because this compound has previously been reported to strongly inhibit in vitro phagocytosis (Rampersad G. et al. op. cit.) and to bind irreversibly to the immunoglobulin anti-D (Shulman, I A et al. op. cit.). In the results presented herein, proof-of-concept has been demonstrated, in that, when combining thimerosal with two different sources of anti-D, a highly significant enhancement effect of thimerosal treatment on the ability of anti-D to inhibit FcγR-mediated phagocytosis is observed. This effect was not attributed to residual, unbound thimerosal nor was it attributed to toxic effects on cell viability.

The mechanism of action of thimerosal treatment to enhance the efficacy of anti-D has not been studied. Thimerosal is known to oxidize free sulfhydryl residues (see, for example, Wu, X. et al. Thiol-Modulated Mechanisms of the Cytotoxicity of Thimerosal and Inhibition of DNA Topoisomerase IIα Chem. Res. Toxicol. 2008 21(2), 483-493).

It can be concluded that thimerosal is likely reacting with the immunoglobulin component of the anti-D preparations as opposed to other excipient protein components because slide-rapid tube anti-D has a much higher protein content (i.e., 30% albumin, package insert) than WINRHO® SDF anti-D and both preparations were affected similarly by chemical treatment. One hypothesis is that the highly reactive thimerosal dissociates to release an ethylmercury and a thiolate anion that is capable of breaking disulfide bonds in the immunoglobulin hinge region. This could result in the thiolate covalently associating with the hinge region sulfurs by creating a new disulfide bond. This may also explain why thimerosal treatment worked very well with anti-D but not with WIG. It is known that the IgG subclass distribution of WIG is comparable to the physiologic distribution (Knezevic-Maramica I, Kruskall M. Intravenous immune globulins: an update for clinicians. Transfusion 2003; 43: 1460-72), whereas anti-D preparations tend to have a higher concentration of IgG3 (Ahaded A, Debbia M, Beolet M, LePennec P Y, Lambin P. Evaluation by enzyme-linked immunosorbent assay of IgG anti-D and IgG subclass concentrations in immunoglobulin preparations. Transfusion 1999; 39:515-21). Given that IgG3 has a longer hinge region due to a significantly larger amount of disulfide bonds compared to IgG1, IgG2, and IgG4 (Golub E S. Immunology: a synthesis. Sunderland (MA): Sinauer Associates; 1987. p. 57), this might result in a greater reactivity with thimerosal for anti-D preparations compared to IVIG. This mechanism would allow the thimerosal to associate covalently with the anti-D preparations and, after the anti-D with the attached thimerosal binds to the monocyte FcγR, blockade may be enhanced through steric effects due to the attached thimerosal. It is also possible that disrupting the hinge region disulfide component of immunoglobulin, particularly IgG3, would possibly alter the affinity of the immunoglobulin Fc for the FcγR. IgG3 anti-D already has increased affinity for FcγR (Kumpel B M, Hadley A G. Functional interactions of red cells sensitized by IgG1 and IgG3 human monoclonal anti-D with enzyme-modified human monocytes and FcR bearing cell lines. Mol Immunol 1990; 27:247-56), and this fact may become even more important if the affinity is increased further by interaction of thimerosal within the hinge region.

In contrast to chemical treatment of anti-D, treatment of IVIG with thimerosal did not significantly enhance the efficacy of IVIG to inhibit FcγR-mediated phagocytosis in vitro. These differences to enhance FcγR blockade when treating anti-D and IVIG immunoglobulin preparations with thimerosal may support the well-accepted understanding that anti-D and IVIG preparations have different mechanisms of action (Song S, Crow A R, Siragam V, Freedman J, Lazarus A H. Monoclonal antibodies that mimic the action of anti-D in the amelioration of murine ITP act by a mechanism distinct from that of IVIg. Blood 2005; 105:1546-8; Cooper N, Heddle N M, Haas M, Reid M E, Lesser M L, Fleit H B, Woloski B M, Bussel J B. Intravenous (IV) anti-D and IV immunoglobulin achieve acute platelet increases by different mechanisms: modulation of cytokine and platelet responses to IV anti-D by FcgammaRIIa and FcgammaRIIIa polymorphisms. Br J Haematol 2004; 124: 511-8).

Thimerosal is an organomercurial compound and is unlikely to be used in humans (Broussard L A, Hammett-Stabler C A, Winecker R E, Ropero-Miller J D. The toxicology of mercury. Lab Med 2002; 33: 614-25; Bigham M, Copes R, Srour L. Exposure to thimerosal in vaccines used in Canadian infant immunization programs, with respect to risk of neurodevelopmental disorders. Can Commun Dis Rep 2002; 28:69-80). Thus, thimerosal has been used for proof-of-concept only, and further studies will be needed to address whether other compounds that inhibit phagocytosis in vitro can be used to treat anti-D with similar results as those presented herein with thimerosal.

When performing these studies, it was observed that the inhibition of FcγR-mediated phagocytosis appeared to require much less anti-D than IVIG. In vivo, it is well known that low-dose anti-D is superior to high-dose IVIG for treatment of ITP and, although the mechanism is unknown, it is not due to increased immunoglobulin aggregates contained in anti-D preparations compared to IVIG (Doman C, Thorpe S J, Thorpe R. Enhanced efficacy of anti-D immunoglobulin for treating ITP is not explained by higher immunoglobulin polymer content. Biologicals 2001; 29:75-9). One explanation for this is the simplified nature of the MMA working via FcγR blockade and the distinct commercial selection methods of anti-D compared to IVIG. For example, anti-D preparations are processed from donors selected as having very high titers of IgG-specific anti-D, and these donors have often had their anti-D titers boosted by multiple deliberate immunizations. In contrast, IVIG is processed from donors selected for the absence of anti-D, but may contain small concentrations of various IgG molecules specific for a multitude of antigens (Sewell W A, Jolles S Immunomodulatory action of intravenous immunoglobulin. Immunology 2002; 107:387-93). Without being bound by theory, it is proposed that deliberate immunization to produce high-titer anti-D results in a maturation of this immune response that not only produces increased affinity maturation of the Fab portion of the molecule but may also produce changes in the Fc portion that result in higher affinity binding to FcγR; this may depend on the subclass of IgG produced (Nimmerjahn F, Ravetch J V. Divergent immunoglobulin G subclass activity through selective Fc receptor binding. Science 2005; 310:1510-2). Hence, the concentration of IgG molecules likely to interact with high affinity and block activating FcγRs in the MMA may be much higher in the anti-D preparations than those of IVIG. This hypothesis was tested by comparing the ability of WINRHO® SDF anti-D to inhibit in vitro FcγR-mediated phagocytosis based on its total immunoglobulin content which is, on average, approximately 100 mg per mL (range, 25-180 mg/mL; M. Genereux, Cangene Corp., Winnipeg, Manitoba, Canada, personal communication, 2006). When using the concentration of total immunoglobulins as 100 mg per mL instead of the anti-D concentration of 300 μg per mL, there was a profound difference in the ability of WINRHO® SDF anti-D to inhibit phagocytosis compared to WIG. Indeed, comparison of titration studies based on the total immunoglobulin concentration of anti-D and WIG found that to inhibit phagocytosis in vitro by 80 and 30 percent, respectively, 300- to 500-fold more IVIG than WINRHO® SDF anti-D was required (Table 1). This result supports the hypothesis that WINRHO® SDF anti-D is much better at blocking the FcγR in vitro than is IVIG. These findings may help to explain the lower dose requirements of anti-D compared to IVIG that are used to treat ITP; 50 μg per kg anti-D compared to 2000 μg per kg WIG is usually required. This hypothesis, however, cannot explain why anti-D preparations would not work in D− individuals, although there have not been many D− ITP patients treated with anti-D and anti-D failures occur even in D+ITP patients (Salama A, Kiefel V, Mueller-Eckhardt C. Effect of IgG anti-Rho(D) in adult patients with chronic autoimmune thrombocytopenia. Am J Hematol 1986; 22:241-50; Rossi E, Damasio E E, Cerri R, Sogno G, Lercari G, Incagliato M, Marmont A. Rhesus antibody treatment for idiopathic thrombocytopenic purpura in an Rh-negative patient. Haematologica 1988; 73:521-3). Indeed, this difference in dosage required between the two preparations has been attributed to other factors (Lazarus A H et al. op. cit.; Song S, Crow A R, Freedman J, Lazarus A H. Monoclonal IgG can ameliorate immune thrombocytopenia in a murine model of ITP: an alternative to IVIg. Blood 2003; 101: 3708-12); however, at least in vitro, this difference may simply reflect a greatly increased ability of anti-D to result in FcR blockade.

TABLE 1
Comparison of Efficiency of WINRHO ® SDF Anti-D to IVIG
for Inhibition of FcγR-mediated Phagocytosis
Amount of total immuglobulin required
(μg/mL)

| Percentage | | | |
|---|---|---|---|
| Inhibition | IVIG | WINRHO ®* | Fold Difference |
| 80 | 100 | 0.33 | 303 |
| 30 | 10 | 0.02 | 500 |

*Based on 100 mg per mL total immunoglobulin in a preparation that contains 300 μg per mL (1500 IU) anti-D (M. Genereux, Cangene Corp., Winnipeg, Manitoba, Canada, personal communication).

In summary, these results indicate that it is possible to link chemical compounds to anti-D to enhance the efficacy of immunoglobulins to inhibit FcγR-mediated phagocytosis. Thus, chemically treated anti-D and IVIG, may become more efficacious at inhibiting phagocytosis in vitro than immunoglobulins used alone.

As noted above, the mechanism of action of thimerosal on the ability of the immunoglobulin anti-D to inhibit FcγR-mediated phagocytosis is unknown. It was thought that this effect may be due to interactions of the thimerosal with the interchain disulfide bonds of the immunoglobulin. In light of this, the inventor wished to determine whether reducing the disulfide bonds of immunoglobulin inhibitors of FcγR-mediated phagocytosis may also produce immunoglobulins having enhanced inhibitory activity.

Thus, in one embodiment, the invention provides a pharmaceutical composition for inhibiting FcγR-mediated phagocytosis comprising a reduced immunoglobulin inhibitor of FcγR-mediated phagocytosis in combination with a pharmaceutically acceptable carrier.

In another embodiment, the invention provides a method for treating or preventing an autoimmune or alloimmune disease comprising administering to a subject in need thereof a therapeutically effective amount of a reduced immunoglobulin inhibitor of FcγR-mediated phagocytosis.

In yet another embodiment, the invention provides a method for inhibiting tissue destruction due to an autoimmune disease comprising administering to a subject in need thereof a therapeutically effective amount of a reduced immunoglobulin inhibitor of FcγR-mediated phagocytosis.

In still another embodiment, the invention provides an immunoglobulin preparation comprising reduced anti-D or reduced IVIG for inhibiting FcγR-mediated phagocytosis.

In another embodiment, the invention provides a method for inhibiting FcγR-mediated phagocytosis in a FcγR-expressing phagocytic cell comprising exposing said phagocytic cell to a reduced immunoglobulin inhibitor of FcγR-mediated phagocytosis.

In another embodiment, the invention provides a method for increasing inhibitory activity of an immunoglobulin inhibitor of FcγR-mediated phagocytosis comprising subjecting said immunoglobulin inhibitor of FcγR-mediated phagocytosis to disulfide reduction.

The term “reduced immunoglobulin inhibitor of FcγR-mediated phagocytosis” refers to an immunoglobulin inhibitor of FcγR-mediated phagocytosis that has been subjected to disulfide reduction. Methods for reducing the disulfide bonds of such immunoglobulins are outlined below. Immunoglobulin inhibitors of FcγR-mediated phagocytosis may include anti-D, WIG, as well as monoclonal antibodies capable of inhibiting FcγR-mediated phagocytosis.

“A therapeutically effective amount” means the amount of a compound that, when administered to a subject for treating or preventing a disease, is sufficient to effect such treatment or prevention for the disease. Those of skill in the art will understand that the “therapeutically effective amount” may vary depending on the compound, the disease and its severity, and the age, weight, etc., of the subject to be treated. In one embodiment of the invention, the subject is a mammal. In another embodiment, the subject is a human.

Two different thiol-containing compounds were used to reduce anti-D: Dithiothreitol (DTT) is a well characterized dual thiol-containing compound that can quantitatively and irreversibly reduce disulfide bonds (Cleland W W. Dithiothreitol, a New Protective Reagent for SH Groups. Biochemistry 1964; 3:480-482) p-toluenesulfonylmethyl mercaptan is also a thiol-containing compound (Langler R F, MacQuarrie S L, McNamara R A, O'Connor P E. A new Synthesis for Anti-fungal α-sulfone Disulfides, Aust J Chem 1999; 52:1119-21). These two thiol-containing compounds were used to treat anti-D in order to break interchain disulfide bonds and then test the efficacy of the chemically-modified anti-D as compared to unmodified anti-D for ability to cause FcγR blockade of mononuclear phagocytes for opsonized red cells.

### Cells, Chemicals and Immunoglobulin

Human whole blood was obtained from volunteers after informed consent as per a Canadian Blood Services Research Ethics Board approved protocol #2005.02. Peripheral blood mononuclear cells (PBMCs) were isolated through density gradient separation using Ficoll-Paque Plus (GE Life Sciences). Dithiothreitol (DTT) and iodoacetamide were purchased from Sigma-Aldrich, Oakville, ON, Canada. p-toluenesulfonylmethyl mercaptan was synthesized in the laboratory of Dr. Richard Langler (Mount Allison University, Sackville, New Brunswick) as previously described (Langler R F et al. ibid.). The sublimed compound was shown to be homogeneous by gas chromatography-mass spectrometry (Langler R F et al. ibid.; Rampersad G, et al. op. cit.). Rh-positive (R₂R₂) red blood cells (RBCs) were provided by Canadian Blood Services and kept in Alsevers solution. Human polyclonal anti-D for Slide and Tube reagent tests was a gift of Marilyn Moulds (Immucor, Houston, Tex., USA) and Dr. Tom Frame (Immucor, Atlanta, Ga., USA).

Chemical Treatment of Immunoglobulin Anti-D with DTT and p-Toluenesulfonylmethyl Mercaptan

The test concentration of anti-D was determined after a titration with the immunoglobulin alone using an MMA as previously described. By this method, a concentration that gave a half-maximum (50%) inhibition value was selected. This half-maximum concentration was a ⅙ dilution for the immunoglobulin anti-D. For each chemical, a concentration of 10⁻² M was initially chosen based on the recommended concentration for the disruption of the interchain disulfide bonds contained in IgM immunoglobulin (Technical Manual, 14^{th} Edition, American Association of Blood Banks, Bethesda, Md. 2002; pp 700-01). DTT was dissolved in phosphate buffered saline (PBS), pH 8.0 to optimize its ability to reduce disulfide bonds irreversibily (Cleland W W et al. op. cit.; Branch D R, Muensch H A, Sy Siok Hian A L, Petz L D. Disulfide bonds are a requirement for Kell and Cartwright (Yt^{a}) blood group antigen integrity. Br J Haematol 1983; 54:573-8). Controls consisted of DTT and anti-D alone, and a mixture of anti-D and DTT. For experiments involving p-toluenesulfonylmethyl mercaptan, the controls and concentrations were initially the same as for DTT (other concentrations were also tested in subsequent experiments, as noted below); however, p-toluenesulfonylmethyl mercaptan was first brought into solution in dimethyl sulfoxide (DMSO) and then titrated to the final concentration with PBS pH 7.4, as previously described (Rampersad G, et al. op. cit.). The final dilution was gently heated to induce all of the p-toluenesulfonylmethyl mercaptan to move into solution prior to testing. The mixture was allowed to cool to room temperature before the immunoglobulin was added. Solutions containing chemical alone, anti-D alone or chemical plus anti-D were placed in separate tubes (Sarstedt) and incubated at 37° C. for 30 minutes at 5% CO₂. The solutions were then deposited in separate cellulose dialysis tubing membranes having a molecular size cut-off of 12,000 daltons (Sigma-Aldrich) as previously described (Rampersad G, et al. ibid.). Dialysis was then done in large 1 L beakers of PBS pH 7.4, for 5-7 days with daily changes of PBS. In separate experiments, in addition to the treatment with the thiol-containing compounds, 5 mM iodoacetamide was added to the mixture of antibody and thiol-containing compounds and incubated for 30 min at room temperature prior to dialysis. The iodoacetamide was used to alkylate the resulting reduced −SH moieties to insure that reduced disulfide bonds would not become oxidized (Virden R, Watts D C. The role of thiol groups in the structure and mechanism of action of arginine kinase. Biochem J 1966; 99:162-72). For experiments involving DTT, the dialysis was held at 4° C., while experiments involving p-toluenesulfonylmethyl mercaptan were done at room temperature to prevent precipitation of the compound out of solution.

While one particular process for producing reduced anti-D has been provided above, it is contemplated that other suitable reducing agents may be substituted for DTT and p-toluenesulfonylmethyl mercaptan, such as, for example, β-mercaptoethanol, dithioerythritol, and glutathione. Essentially, suitable reducing agents for reducing anti-D and other immunoglobulin inhibitors of FcγR-mediated phagocytosis include any compounds containing at least one thiol group. Reduced anti-D may also be prepared according to known methods as outlined in U.S. Pat. No. 4,296,090 to Ortho Diagnostics, Inc., the contents of which are herein incorporated by reference in this regard.

While iodoacetamide is used above as the S-alkylating agent, other alkylating agents may be used, as outlined in U.S. Pat. No. 4,296,090 to Ortho Diagnostics, Inc., the contents of which are herein incorporated by reference in this regard. For example, iodoacetic acid or other equivalent methods of preventing disulfide reformation may also be used.

While dialysis is used to separate the reduced anti-D from the reducing agent and byproducts thereof, it is contemplated that other methods of isolating the reduced anti-D from the reducing agent and byproducts thereof may be used, such as affinity chromatography, High Performance Liquid Chromatography (HPLC), gel filtration chromatography, and centrifugal concentrators (such as CENTRICON® centrifugal concentrators).

Following dialysis, a MMA was used to compare the ability of each solution, against an unmanipulated control PBS solution (for DTT) or DMSO-containing control solution (for p-toluenesulfonylmethyl mercaptan), for their ability to inhibit FcγR-mediated phagocytosis of anti-D sensitized R₂R₂ RBCs. Dialysed chemical alone was tested to insure all free chemical was dialysed compared to an undialyzed control chemical. The effect of dialyzed anti-D alone or chemically-treated anti-D were also tested for ability of each to inhibit monocyte-macrophage phagocytosis.

Red Blood Cell Sensitization and MMA Assay with DTT- and p-Toluenesulfonylmethyl Mercaptan-Treated Anti-D

Preparation of sensitized R₂R₂ RBCs and MMA assay were done exactly as previously described. In brief, for the MMA, after attachment of the monocytes to coverslips, the coverslips were washed vigorously in PBS to remove non-adherent cells. The coverslips were then overlayed with medium alone (control of maximum phagocytosis) or with one millilitre of undialyzed DTT, dialyzed DTT, undialyzed p-toluenesulfonylmethyl mercaptan, or dialyzed p-toluenesulfonylmethyl mercaptan, or with dialyzed anti-D that had been treated with DTT or p-toluenesulfonylmethyl mercaptan. The coverslips were then incubated at 37° C. and 5% CO₂ for 1 hour and subsequently washed again in PBS to remove excess chemical or immunoglobulin. One millilitre of opsonised RBC solution was then layered onto the coverslips and they were then incubated again for 2 hours at 37° C. and 5% CO₂. Coverslips were fixed and evaluated under phase-contrast microscopy (Rampersad G, et al. op. cit.; Foo A H et al. op. cit.).

### Analysis and Statistics

A phagocytic index was calculated as previously described (Rampersad G, et al. op. cit.; Foo A H et al. op. cit.), as the unitless number of phagocytosed red cells per 100 macrophages. A typical phagocytic index for anti-D sensitized R₂R₂ red blood cells was 90-100. Residual, attached, and phagocytosed RBCs were distinguishable by relative differences in refracted light under phase-contrast microscopy. Percent inhibition was calculated as previously described (Rampersad G, et al. op. cit.; Foo A H et al. op. cit.) taking the phagocytic control index, using only culture media, to be the maximum phagocytosis possible equal to 100 percent. The means and standard error of the mean (SEM) of the results of several independent experiments were determined and analysed using Student's t-test.

Effect of DTT and p-Toluenesulfonylmethyl Mercaptan Treatment of Anti-D on Inhibition of Phagocytosis

DTT or p-toluenesulfonylmethyl mercaptan (see FIG. 5 for chemical structures of these chemicals and thimerosal) at 10⁻² M was combined with slide/rapid tube anti-D used at a ⅙ dilution to give a half-maximum (50%) inhibitory effect when used without chemical modification. As shown in FIGS. 6 and 7A, anti-D at a dilution of ⅙ after extensive dialysis was able to retain its ability to inhibit subsequent phagocytosis of opsonized red cells by approximately 50%. However, when either DTT (FIG. 6) or p-toluenesulfonylmethyl mercaptan (FIG. 7A) was used to reduce the disulfide bonds of the anti-D, the ability of the chemically-modified immunoglobulin to inhibit phagocytosis increased, from 50% inhibitory activity to 100%. This increase in efficacy is not attributed to free DTT or p-toluenesulfonylmethyl mercaptan, as shown in FIGS. 6 and 7A, as extensive dialysis removes any free compound. Indeed, free DTT at 10⁻²M will inhibit phagocytosis by about 30%; however, after dialysis there is no inhibition using DTT alone, illustrating that free DTT was completely dialyzed out of the tubing. p-toluenesulfonylmethyl mercaptan has previously been shown to inhibit FcγR-mediated phagocytosis by approximately 70% at a concentration of 10⁻⁴M using a MMA (Rampersad G, et al. op. cit.); however, after dialysis at room temperature, p-toluenesulfonylmethyl mercaptan was not able to inhibit phagocytosis, illustrating that it was completely dialysed. In addition, it has also been demonstrated previously that chemically-modified anti-D after dialysis does not result in increased cellular toxicity. Although iodoacetamide was also utilized to alkylate free sulfhydryls, this was not required to achieve a 100% inhibitory effect at the concentrations of DTT or p-toluenesulfonylmethyl mercaptan noted above.

At concentrations of p-toluenesulfonylmethyl mercaptan of 10⁻⁵M and 10⁻⁶M, the treated anti-D has about 50% ability to inhibit phagocytosis of anti-D-coated R₂R₂ cells, while if the p-toluenesulfonylmethyl mercaptan-treated anti-D has been S-alkyated with iodoacetamide, this improves the inhibitory ability to about 80% and 85%, respectively (FIG. 7B). S-alkylation has no effect on the ability of anti-D treated with p-toluenesulfonylmethyl mercaptan at a concentration of 10⁻²M to inhibit FcγR-mediated phagocytosis. The reason for this is that the concentration of p-toluenesulfonylmethyl mercaptan is so high that the equilibrium is shifted towards the reduced state without necessitating S-alkylation. These experiments prove that reduction of disulfide bonds is the critical process and that S-alkylation is necessary (at low concentrations and for in vivo work) to maintain the disulfide bonds in their reduced state.

Fluorescence-Activated Cell Sorting (FACS) Analysis for Viable and Apoptotic Cells Following Incubation with Anti-D, Dtt-Treated Anti-D, and p-Toluenesulfonylmethyl Mercaptan-Treated Anti-D

The toxicity of chemically-modified anti-D was tested using primary PBMNC-derived adherent monocytes after incubation with untreated, DTT- or p-toluenesulfonylmethyl mercaptan-treated anti-D. Effects of treatment were assessed with dual-color flow cytometry with PI and annexin V-FITC (FIG. 8). Tests utilized untreated monocytes and monocytes treated with dialyzed slide-rapid tube anti-D alone, used at a 1-in-6 dilution, or dialyzed anti-D that had been previously mixed with DTT or p-toluenesulfonylmethyl mercaptan at 10⁻² mol per L. Annexin V-FITC fluorescence is represented on the horizontal axis and PI fluorescence is shown on the vertical axis. The viable, early apoptotic, late apoptotic, and necrotic cells are found in the lower left, lower right, upper right, and upper left quadrants, respectively. Percentage of cells within each quadrant is indicated. These results indicate that there is no cellular toxicity due to residual reducing chemical or the reduced immunoglobulin as there is no significant increase in apoptosis (using Annexin V-FITC) or dead cells (using propidium iodide staining) compared to untreated anti-D.

### Prophetic Example

Using a mouse model of anti-D therapy for ITP (Song S, Crow A R, Siragam V, Freedman J, Lazarus A H. Monoclonal antibodies that mimic the action of anti-D in the amelioration of murine ITP act by a mechanism distinct from that of IIg. Blood. 2005 Feb. 15; 105(4):1546-8. Epub 2004 Oct. 12) it is expected that reduction of the therapeutic antibody will increase its efficacy to reverse immune thrombocytopenia. The mouse model consists of CD1 mice that are administered anti-platelet antibody (anti-CD41) daily starting at day 0. By day 1, the mouse platelet numbers drop to a nadir and remain low over time. At day 2, an antibody to mouse red blood cells (anti-TER-119) is administered to one set of mice and one set of mice remains untreated. By day 4, the mouse platelet numbers are expected to increase to normal levels only in the anti-TER-119-treated mice, despite daily administration of anti-platelet antibody. The dose-response for reversal of the platelet destruction will be determined. It is expected that the ability of the anti-TER-119 to reverse the platelet destruction will be increased following its treatment with DTT, thimerosal or p-toluenesulfonylmethyl mercaptan, S-alkylation, and extensive dialysis. The comparison of the effect on reversal of the anti-platelet mediated destruction is monitored by titration comparison of the efficacy of untreated and reduced anti-TER-119.

Discussion of Results Obtained with DTT- and p-Toluenesulfonylmethyl Mercaptan-Treated Anti-D

Herein it has been demonstrated that it is possible to chemically modify anti-D so that the ability of the modified anti-D to inhibit FcγR-mediated phagocytosis is greatly improved. In this work, thimerosal was used to show proof-of-concept in vitro; however, thimerosal is a controversial compound that contains mercury and likely would never be used in any clinical application (Broussard L A et al. op. cit.; Health Canada. Exposure to Thimerosal in Vaccines used in Canadian Infant Immunization Programs, With Respect to Risk of Neurodevelopmental Disorders. Canada Communicable Disease Report 2002; 28:69-80). Furthermore, the mechanism of the thimerosal effect on anti-D was unknown. It was speculated that the highly reactive thimerosal dissociates to release an ethylmercury and a thiolate anion that is capable of breaking disulfide bonds in the immunoglobulin hinge region. This could result in the thiolate covalently associating with the hinge region sulfurs by creating a new disulfide bond. It was further speculated that the reason thimerosal was able to increase the efficacy for FcγR blockade when using anti-D compared to IVIg might be due to an increased level of IgG3 contained in hyperimmune anti-D serum compared to non-hyperimmune IVIg (Ahaded A et al. op. cit.). IgG3 contains more disulfide linkages between the two heavy chains than any other IgG subtype (Golub E S op. cit.). In order to determine if disulfide bonds of anti-D immunoglobulin may be targeted, well characterized thiol-containing compounds that are known to reduce disulfide bonds have now been tested.

It was found that DTT, which is well characterized as a dual thiol-containing compound (FIG. 5) with unusual chemical characteristics that allows it to irreversibly reduce disulfide bonds without alkylation (Cleland W W et al. op. cit.; Branch D R et al. 1983 op. cit.), was able to greatly enhance the ability of anti-D to inhibit FcγR-mediated phagocytosis in a manner similar to that of thimerosal. Likewise, another thiol-containing compound, p-toluenesulfonylmethyl mercaptan, also was able to enhance the ability of anti-D to block FcγR-mediated phagocytosis. Although treatment with p-toluenesulfonylmethyl mercaptan should result in reversible reduction of disulfide bonds, alkylation was not required to see the effects of treatment with this compound. Perhaps this was due to the high concentration, 10⁻²M, of p-toluenesulfonylmethyl mercaptan used in these studies, forcing the equilibrium to reduction of disulfide bonds. Thus, it may be possible to greatly reduce the treatment concentrations of thiol-compounds in order to achieve the same effect, using alkylation to maintain the disulfide bonds in a reduced state. Alkylation, due to prevention of oxidation back to a disulfide, may also improve the stability of the anti-D to maintain its enhanced effect on FcγR blockade. In practice, the step of reduction of the immunoglobulin inhibitors of FcγR-mediated phagocytosis would preferably be followed by S-alkylation for in vivo use of these inhibitors. As noted above, S-alkylation allows for lower doses of reducing compound to be used in order to obtain the same increased efficacy, as S-alkylation maintains the interchain disulfide bonds in a reduced state.

Based on these current results, it is believed that the breakage of the disulfide bonds within the hinge region of the immunoglobulin causes the antibody to attain increased flexibility and/or affinity to certain FcγR receptors. Perhaps, the chemical modification allows the FcγR to crosslink and activate the inhibitory FcγRIIB receptors (Samuelsson A, Towers T L, Ravetch N. Anti-inflammatory activity of IVIG mediated through the inhibitory Fc receptor. Science 2001; 291:484-6) and/or more efficiently block FcγRI activating receptors? Without being bound by theory, it is also postulated that the increased flexibility of the antibody may allow a single antibody to interact with two FcγR receptors, wherein each of its Fc heavy chains would interact with a FcγR receptor.

In summary, these results show that breaking disulfide bonds within anti-D will increase the molecules' ability to result in FcγR blockade of monocytes in vitro. These results indicate that strategies to chemically modify anti-D do not require mercury-containing compounds, such as thimerosal, and provides rationale to further explore this effect in order to reduce the amount of anti-D required for effective therapy of ITP and, in turn, reduce the possible side effects of this therapy.

In light of the widespread use of immunoglobulin inhibitors of FcγR-mediated phagocytosis as therapeutic agents, and in view of the promising in vitro results shown herein for reduced anti-D, it is fully expected that reduced immunoglobulin inhibitors of FcγR-mediated phagocytosis will have utility in the treatment/prevention of autoimmune or alloimmune diseases. For instance, reduced immunoglobulin inhibitors of FcγR-mediated phagocytosis may be used to treat autoimmune or alloimmune diseases selected from the group consisting of immune thrombocytopeniac purpura, autoimmune haemolytic anemias, alloimmune haemolytic anemias, haemolytic transfusion reaction, haemolytic disease of the newborn, alloimmune neutropenia, autoimmune neutropenia, drug-induced haemolytic anemias, and immune cytopenia. It is envisioned that any kind of immune-mediated disease where the mechanism involves FcγR-mediated phagocytosis could be treated with reduced immunoglobulin inhibitors of FcγR-mediated phagocytosis. Reduced immunoglobulin inhibitors of FcγR-mediated phagocytosis may also be used to inhibit tissue destruction due to an autoimmune disease such as rheumatoid arthritis, multiple sclerosis, and myasthenia gravis, for example.

Immunoglobulin inhibitors of FcγR-mediated phagocytosis, such as anti-D, are typically administered intravenously. Suitable pharmaceutically acceptable carriers are known to those of skill in the art.

Dose requirements of immunoglobulin inhibitors of FcγR-mediated phagocytosis will depend on the disease that is being treated, and such dose requirements are known to those of skill in the art. As noted above, dose requirements of anti-D that are used to treat up are generally around 50 μg per kg anti-D. In light of the in vitro results provided herein, it is expected that dose requirements of reduced immunoglobulin inhibitors of FcγR-mediated phagocytosis will be generally around half of the required doses for their non-reduced counterparts.

While the above studies relate to reduced anti-D and its use as an inhibitor of FcγR-mediated phagocytosis, it is contemplated that other reduced immunoglobulin inhibitors of FcγR-mediated phagocytosis may also be used. As noted above, although the inhibitory activity of IVIG alone and thimerosal-treated IVIG did not significantly differ according to t test, ANOVA, general linear model, or Student-Newman-Keuls t tests, a trend of enhanced ability of IVIG to inhibit phagocytosis in vitro after chemical to treatment was observed (see FIG. 4). It is expected that a similar trend will be exhibited for reduced IVIG and for reduced and S-alkylated IVIG. Furthermore, human monoclonal antibodies have been shown to be inhibitors of FcγR-mediated phagocytosis (see U.S. Pat. No. 5,851,524) and it is expected that reduction of these antibodies may also increase their inhibitory activity.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

The embodiments of the invention described above are intended to be exemplary only. The scope of the invention is therefore intended to be limited solely by the scope of the appended claims

Every reference cited herein is hereby incorporated by reference in its entirety.

What is claimed is:

1. A method for treating or preventing an autoimmune or alloimmune disease comprising administering to a subject in need thereof a therapeutically effective amount of a reduced immunoglobulin inhibitor of FcγR-mediated phagocytosis.

2. The method of claim 1, wherein said autoimmune or alloimmune disease is selected from the group consisting of immune thrombocytopeniac purpura, autoimmune haemolytic anemias, alloimmune haemolytic anemias, haemolytic transfusion reaction, haemolytic disease of the newborn, alloimmune neutropenia, autoimmune neutropenia, drug-induced haemolytic anemias, and immune cytopenia.

3. The method of claim 1, wherein said reduced immunoglobulin inhibitor of FcγR-mediated phagocytosis comprises reduced anti-D.

4. The method of claim 1, wherein said reduced immunoglobulin inhibitor of FcγR-mediated phagocytosis comprises reduced IVIG.

5. The method of claim 1, wherein said reduced immunoglobulin inhibitor of FcγR-mediated phagocytosis comprises a reduced monoclonal antibody.

6. The method of claim 1, wherein said reduced immunoglobulin inhibitor of FcγR-mediated phagocytosis is S-alkylated.

7. A method for inhibiting tissue destruction due to an autoimmune disease comprising administering to a subject in need thereof a therapeutically effective amount of a reduced immunoglobulin inhibitor of FcγR-mediated phagocytosis.

8. The method of claim 7, wherein said autoimmune disease is selected from the group consisting of rheumatoid arthritis, multiple sclerosis, and myasthenia gravis.

9. The method of claim 7, wherein said reduced immunoglobulin inhibitor of FcγR-mediated phagocytosis comprises reduced anti-D.

10. The method of claim 7, wherein said reduced immunoglobulin inhibitor of FcγR-mediated phagocytosis comprises reduced IVIG.

11. The method of claim 7, wherein said reduced immunoglobulin inhibitor of FcγR-mediated phagocytosis comprises a reduced monoclonal antibody.

12. The method of claim 7, wherein said reduced immunoglobulin inhibitor of FcγR-mediated phagocytosis is S-alkylated.

13. A method for inhibiting FcγR-mediated phagocytosis in a FcγR-expressing phagocytic cell comprising exposing said phagocytic cell to a reduced immunoglobulin inhibitor of FcγR-mediated phagocytosis.

14. The method of claim 13, wherein said reduced immunoglobulin inhibitor of FcγR-mediated phagocytosis comprises reduced anti-D.

15. The method of claim 13, wherein said reduced immunoglobulin inhibitor of FcγR-mediated phagocytosis comprises reduced IVIG.

16. The method of claim 13, wherein said reduced immunoglobulin inhibitor of FcγR-mediated phagocytosis comprises a reduced monoclonal antibody.

17. The method of claim 13, wherein said reduced immunoglobulin inhibitor of FcγR-mediated phagocytosis is S-alkylated.
